(19) United States
(12) Patent Application Publication
(10) Pub. No.: US 2017/0354988 A1
(43) Pub. Date: Dec. 14, 2017

(54) HUMIDIFIER

(71) Applicant: DAIKIN INDUSTRIES, LTD., Osaka-shi, Osaka (JP)

(72) Inventors: Tsunahiro OHDOU, Osaka (JP); Masaya NISHIMURA, Osaka (JP); Tomoki SAITOU, Osaka (JP); Sachiko YAMAGUCHI, Osaka (JP)

(73) Assignee: DAIKIN INDUSTRIES, LTD., Osaka-shi, Osaka (JP)

(21) Appl. No.: 15/539,363

(22) PCT Filed: Dec. 24, 2015
(86) PCT No.: PCT/JP2015/006464

§ 371 Date: Jun. 23, 2017

(30) Foreign Application Priority Data
Dec. 26, 2014 (JP) 2014-266331

(51) Int. Cl. B05B 17/00; B01F 3/04; F24F 6/12; C02F 1/36; A61L 9/12; A61L 9/14

(52) U.S. Cl. CPC B05B 17/0646; F24F 6/12; C02F 1/36; B01F 3/0407; A61L 9/14; A61L 9/122

(57) ABSTRACT

The humidifier includes a humidification section which vaporizes water to produce humidified air, and a fan which produces in an air passage an air flow delivering the humidified air to the outside. The humidifier further includes a sterilized water producing section which produces sterilized water, an ultrasonic irradiator which produces sterilizing mist through ultrasonic irradiation of the sterilized water, an auxiliary passage through which the sterilizing mist is delivered to the outside, and an operation switching mechanism. The operation switching mechanism is switched between a first operation mode in which the air passage and the auxiliary passage communicate with each other to introduce the sterilizing mist into the air passage, and a second operation mode in which communication between the air passage and the auxiliary passage is blocked.

### TECHNICAL FIELD

The present invention relates to a humidifier, and more particularly relates to a humidifier having a sterilizing function.

### BACKGROUND ART

A humidifier which produces humidified air by allowing the air to pass through a humidification rotor partially immersed in water stored in a reservoir has been known. Purification of water in the reservoir of the humidifier of such a configuration has been proposed to prevent possible growth of bacteria in the water in the reservoir.

For example, Patent Document 1 discloses a humidity controller including a liquid treatment discharge unit arranged in a water tank to purify water stored in the water tank.

### CITATION LIST

### PATENT DOCUMENTS

[Patent Document 1] Japanese Patent No. 4737330

### SUMMARY OF THE INVENTION

### Technical Problem

The humidity controller disclosed by Patent Document 1 vaporizes water purified by the liquid treatment discharge unit with the humidification rotor, and simply releases the vapor into the room. Thus, the humidity controller is less convenient, and is susceptible to improvement.

In view of the foregoing background, it is therefore an object of the present invention to improve the convenience of a humidifier having a sterilizing function.

### Solution to the Problem

To achieve the object described above, an aspect of the present disclosure is directed to a configuration including: an operation switching mechanism (9) which is switched between a first operation mode in which an air passage (5) through which humidified air is delivered to the outside and an auxiliary passage (6) through which sterilizing mist is delivered to the outside communicate with each other to introduce the sterilizing mist into the air passage (5), and a second operation mode in which communication between the air passage (5) and the auxiliary passage (6) is blocked.

A first aspect of the present disclosure is directed to a humidifier including: a humidification section (50) which vaporizes water stored therein to produce humidified air; an air passage (5) through which the humidified air is delivered to the outside; and a fan (60) which produces in the air passage (5) an air flow delivering the humidified air to the outside, wherein the humidifier further includes: a sterilized water producing section (10) which produces a sterilizing factor in water stored therein to produce sterilized water containing the sterilizing factor, an ultrasonic irradiator (72) which atomizes the sterilized water through ultrasonic irradiation of the sterilized water to produce sterilizing mist, an auxiliary passage (6) through which the sterilizing mist is delivered to the outside, and an operation switching mechanism (9) which is switched between a first operation mode in which the air passage (5) and the auxiliary passage (6) communicate with each other to introduce the sterilizing mist to the air passage (5), and a second operation mode in which communication between the air passage (5) and the auxiliary passage (6) is blocked.

According to the first aspect, the fan (60) produces in the air passage (5) an air flow delivering the humidified air produced by the humidification section (50) to the outside. Further, the ultrasonic irradiator (72) atomizes the sterilized water produced by the sterilized water producing section (10) through ultrasonic irradiation of the sterilized water to produce sterilizing mist.

According to the first aspect, while the operation switching mechanism (9) is in the first operation mode, the air passage (5) communicates with the auxiliary passage (6) through which the sterilized water is delivered to the outside so as to introduce the sterilizing mist into the air passage (5). Thus, the sterilizing mist in the auxiliary passage (6) is mixed with the humidified air in the air passage (5). This increases the absolute humidity of the humidified air blown from the air passage (5) into the room. That is, the humidified air containing a large amount of moisture is supplied into the room, thereby humidifying the inside of the room with reliability.

Further, according to the first aspect, while the operation switching mechanism (9) is in the second operation mode, the communication between the air passage (5) and the auxiliary passage (6) through which the sterilized water is delivered to the outside is blocked. Thus, the sterilizing mist in the auxiliary passage (6) is released from the auxiliary passage (6) to the outside. In this state, if hands or articles, for example, are placed in the flow of the sterilizing mist blown from the auxiliary passage (6), the hands or articles may be sterilized.

Thus, according to the humidifier (80) of the first aspect, mixing the sterilizing mist produced by atomization of the sterilized water with the humidified air outside improves the humidification capacity. In addition, the sterilizing mist may be supplied from the auxiliary passage (6) to a relatively narrow region of the room so that the humidifier may be used for various other purposes (e.g., sterilization of hands or articles). Therefore, according to the first aspect, the humidifier may be provided with improved convenience.

A second aspect of the present disclosure is an embodiment of the first aspect of the disclosure. According to the second aspect, the operation switching mechanism (9) includes an outlet valve (7) which allows or blocks communication between an end of the air passage (5) adjacent to an outlet and an end of the auxiliary passage (6) adjacent to an outlet, and an inlet valve (8) which allows or blocks communication between an end of the air passage (5) upstream of the outlet valve (7) and an end of the auxiliary passage (6) adjacent to the ultrasonic irradiator (72), and the fan (60) is configured to produce in the auxiliary passage (6) an air flow delivering the sterilizing mist to the outside.

According to the second aspect, the operation switching mechanism (9) includes the outlet and inlet valves (7) and (8) which allow the air passage (5) and the auxiliary passage (6) to communicate with each other as needed. Opening/closing the outlet and inlet valves (7) and (8) switches the operation switching mechanism (9) between the first and second operation modes. Furthermore, the fan (60), which produces in the air passage (5) the air flow delivering the humidified air to the outside, produces in the auxiliary passage (6) an air flow delivering the sterilizing mist to the outside. Thus, the air flow delivering the humidified air to the outside and the air flow delivering the sterilizing mist to the outside are produced by the single fan (60).

A third aspect of the present disclosure is an embodiment of the second aspect. According to the third aspect, the operation switching mechanism (9) in the first operation mode allows the inlet valve (8) to open, and the outlet valve (7) to open toward the auxiliary passage (6), so that the sterilizing mist in the auxiliary passage (6) is introduced into the air passage (5).

According to the third aspect, the operation switching mechanism (9) in the first operation mode is configured to allow the inlet valve (8) to open, and the outlet valve (7) to open toward the auxiliary passage (6), so that the sterilizing mist in the auxiliary passage (6) is introduced into the air passage (5). The flow of the humidified air introduced via the inlet valve (8) produces the air flow including the sterilizing mist in the auxiliary passage (6), which is then introduced into the air passage (5) via the outlet valve (7). Consequently, the sterilizing mist is mixed with the humidified air to increase the absolute humidity of the humidified air, and the room is reliably humidified by the humidified air blown into the room.

A fourth aspect of the present disclosure is an embodiment of the second aspect. According to the fourth aspect, the operation switching mechanism (9) in the first operation mode allows the inlet valve (8) to open, and the outlet valve (7) to open toward the air passage (5), so that the humidified air in the air passage (5) is introduced into the auxiliary passage (6).

According to the fourth aspect, the operation switching mechanism (9) in the first operation mode allows the inlet valve (8) to open, and the outlet valve (7) to open toward the air passage (5), so that the humidified air in the air passage (5) is introduced into the auxiliary passage (6). Thus, the flow of the humidified air introduced via the inlet valve (8) produces the air flow including the sterilizing mist in the auxiliary passage (6). Furthermore, the air flow including the sterilizing mist in the auxiliary passage (6) is accelerated by the flow of the humidified air introduced via the outlet valve (7). Thus, the sterilizing mist is vigorously blown from the auxiliary passage (6) into the room, thereby bringing the sterilizing mist to a position away from the air purifier (80).

A fifth aspect of the present disclosure is an embodiment of the second aspect. According to the fifth aspect, the operation switching mechanism (9) in the second operation mode allows the inlet valve (8) to open, and the outlet valve (7) to close.

According to the fifth aspect, the operation switching mechanism (9) in the second operation mode allows the inlet valve (8) to open, and the outlet valve (7) to close. Thus, the flow of the humidified air introduced via the inlet valve (8) produces the air flow including the sterilizing mist in the auxiliary passage (6). As a result, the sterilizing mist may be released from the auxiliary passage (6) to a relatively narrow region of the room together with the humidified air.

A sixth aspect of the present disclosure is an embodiment of any one of the first to fifth aspects. According to the sixth aspect, the sterilized water producing section (10) includes: a water treatment vessel (13) which stores water; a pair of electrodes (16, 17) which are provided to be immersed in water in the water treatment vessel (13); and a divider (11) which is made of an electrically insulating material, and divides a space in the water treatment vessel (13) into a portion in which one of the pair of electrodes (16, 17) is immersed in water, and a portion in which the other of the pair of electrodes (16, 17) is immersed in water. A through hole (12h) is formed through the divider (11) to form a current path between the pair of electrodes (16, 17), and electric discharge is caused in the through hole (12h) in water to produce the sterilizing factor.

According to the sixth aspect, the sterilized water producing section (10) includes a water treatment vessel (13) which stores water, a pair of electrodes (16, 17) which are provided to be immersed in water in the water treatment vessel (13), and a divider (11) which divides a space in the water treatment vessel (13) into a portion in which one of the pair of electrodes (16, 17) is immersed in water, and a portion in which the other of the pair of electrodes (16, 17) is immersed in water. The divider (11) has a through hole (12h) which forms a current path between the pair of electrodes (16, 17). Thus, when a voltage is applied between the pair of electrodes (16, 17), a bubble is formed in water stored in the water treatment vessel (13), more specifically, in the through hole (12h) penetrating the divider (11) to serve as the current path between the pair of electrodes (16, 17), and electric discharge occurs in the bubble. As a result, a sterilizing factor, such as hydroxyl radicals, may be produced in the water stored in the water treatment vessel (13). The hydroxyl radicals produced in the water in the water treatment vessel (13) shortly turn to be hydrogen peroxide. However, the hydrogen peroxide is decomposed and hydroxyl radicals are produced again through ultrasonic irradiation by the ultrasonic irradiator (72). Thus, the sterilizing mist containing hydroxyl radicals may be released into the room.

### Advantages of the Invention

According to the aspect of the present disclosure described above, the humidifier includes the operation switching mechanism (9) which is switched between the first operation mode in which the air passage (5) through which the humidified air is delivered to the outside and the auxiliary passage (6) through which the sterilizing mist is delivered to the outside communicate with each other to introduce the sterilizing mist into the air passage (5), and a second operation mode in which communication between the air passage (5) and the auxiliary passage (6) is blocked. This configuration improves the convenience of a humidifier having a sterilizing function.

### BRIEF DESCRIPTION OF THE DRAWINGS

FIG. 1 is a schematic diagram illustrating an internal configuration of an air purifier according to a first embodiment as viewed from the side.

FIG. 2 is a schematic diagram illustrating the internal configuration of the air purifier according to the first embodiment as viewed from the back.

FIG. 3 is a schematic diagram illustrating a water treatment vessel forming part of the air purifier according to the first embodiment.

FIG. 4 is a schematic view illustrating a major part of a divider of a sterilized water producing section of a water treatment unit forming part of the air purifier according to the first embodiment.

FIG. 5 is a schematic view illustrating a water treatment vessel of the sterilized water producing section of the water treatment unit forming part of the air purifier according to the first embodiment, where the water treatment vessel is in a draining state.

FIG. 6 is a schematic view illustrating an operation switching mechanism forming part of the air purifier according to the first embodiment, where the operation switching mechanism is in a second state of a first operation mode.

FIG. 7 is a schematic view illustrating the operation switching mechanism forming part of the air purifier according to the first embodiment, where the operation switching mechanism is in a second operation mode.

FIG. 8 is a schematic view illustrating the operation switching mechanism forming part of the air purifier according to the first embodiment, where the operation switching mechanism is in a third operation mode.

### DESCRIPTION OF EMBODIMENTS

Embodiments of the present invention will now be described in detail with reference to the drawings. Note that the present invention is not limited to the following embodiments.

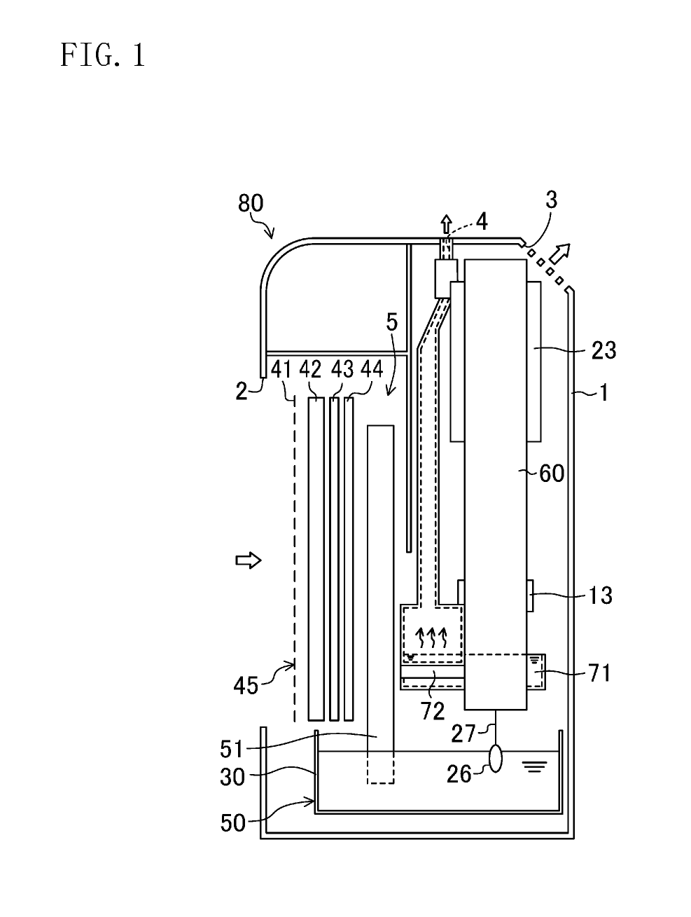

FIGS. 1-9 illustrate an embodiment of a humidifier. In the present embodiment, an air purifier having a humidifying function will be described as an example of the humidifier.

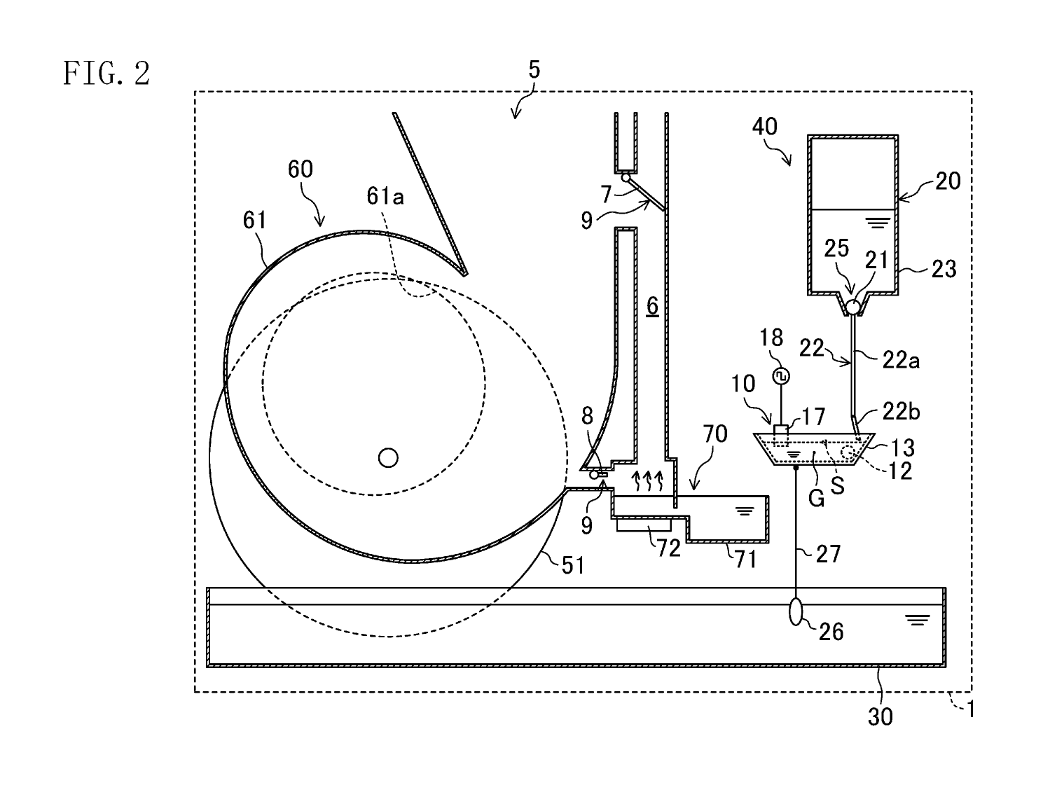

The air purifier (80) includes, as shown in FIGS. 1 and 2, a casing (1) which is made of a resin, and houses an air purification unit (45), a humidification section (50), a water treatment unit (40), an ultrasonic irradiation unit (70), and a fan (60).

<Casing>

The casing (1) has the shape of a rectangular parallelepiped box. As shown in FIG. 1, the casing (1) has an inlet (2) formed through a front surface thereof (a left surface in FIG. 1) through which the air is introduced into the casing (1). The casing (1) also has a primary outlet (3) formed through a rear portion of a top surface thereof as shown in FIG. 1, through which the air in the casing (1) is blown to the inside of a room. The casing (1) also has a secondary outlet (4) formed through the top surface thereof as shown in FIG. 1, through which sterilizing mist produced in the casing (1) is blown into a room. An air passage (5) is formed inside the casing (1) as shown in FIG. 1 to allow the air to flow from the inlet (2) to the primary outlet (3).

As shown in FIG. 1, the air purification unit (45), the humidification section (50), the water treatment unit (40), and the fan (60) are sequentially arranged in the air passage (5) in the direction of the air flow in the air passage (5). A front panel (not shown) is attached to the front surface (left surface in FIG. 1) of the casing (1) so as not to close the inlet (2).

The air passage (5) is a passage through which the air humidified by the humidification section (50) is delivered to the outside. As shown in FIG. 2, the fan (60) is arranged at an end of the air passage (5) adjacent to the primary outlet (3), and a space inside a fan casing (61) (to be described later) of the fan (60) forms part of the air passage (5). Further, as shown in FIG. 2, the fan casing (61) is connected to an auxiliary passage (6) through which sterilizing mist is delivered to the outside.

As shown in FIG. 2, the ultrasonic irradiation unit (70) is provided adjacent to an end of the auxiliary passage (6) connected to the air passage (5). An outlet valve (7) is provided between an end of the air passage (5) adjacent to the primary outlet (3) and an end of the auxiliary passage (6) adjacent to the secondary outlet (4) to allow or block the communication between these passages. An inlet valve (8) is provided between an end of the air passage (5) upstream of the outlet valve (7) and an end of the auxiliary passage (6) adjacent to the ultrasonic irradiation unit (70) to allow or block the communication between these passages. The inlet and outlet valves (7) and (8) constitute an operation switching mechanism (9) which is switched between a first operation mode in which the air passage (5) and the auxiliary passage (6) communicate with each other to introduce the sterilizing mist into the air passage (5), and a second operation mode in which the communication between the air passage (5) and the auxiliary passage (6) is blocked.

<Air Purification Unit>

The air purification unit (45) includes a prefilter (41), an ionizer (42), an electrostatic filter (43), and a deodorizing filter (44) which are sequentially arranged in the direction of the air flow as shown in FIG. 1, and is configured to purify the air flowing through the air passage (5).

The prefilter (41) is configured to trap relatively large dust particles in the air sucked through the inlet of the air passage (5).

The ionizer (42) is configured to allow relatively small dust particles in the air that has passed the prefilter (41) to be positively charged, for example. Further, the ionizer (42) includes a linear electrode and a plate electrode facing each other, for example, and is configured to apply a voltage to the linear and plate electrodes to cause corona discharge between the linear and plate electrodes.

The electrostatic filter (43) is configured to electrically attract and trap the dust particles charged by the ionizer (42).

The deodorizing filter (44) supports a deodorant for deodorization of the air on a surface of a honeycomb base material. The deodorant may be an adsorbent which adsorbs target components (odor and toxic substances) in the air, a catalyst which oxidatively decomposes the target components, or any other suitable material.

<Water Treatment Unit>

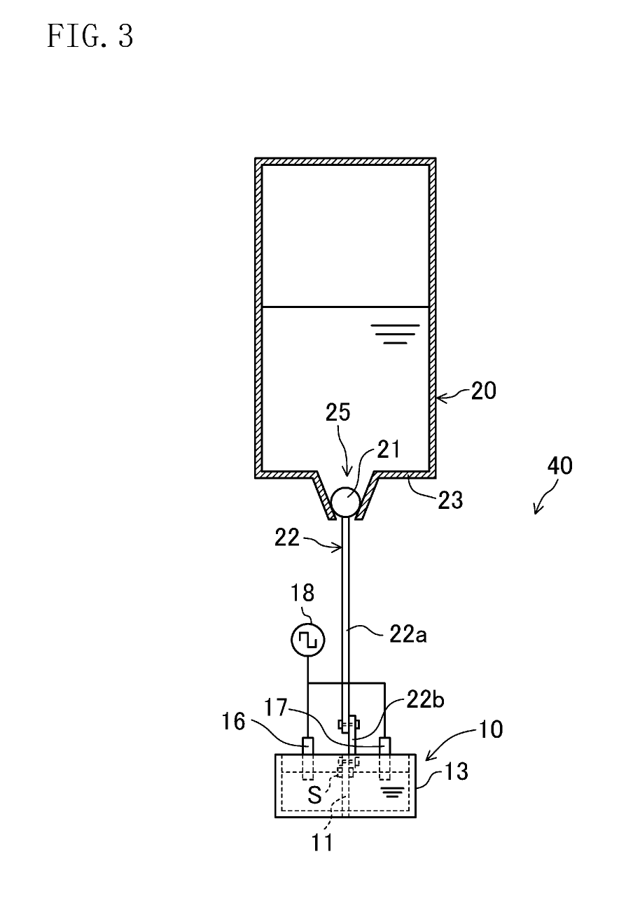

The water treatment unit (40) includes, as shown in FIG. 3, a sterilized water producing section (10) which produces a sterilizing factor in water stored therein to produce sterilized water containing the sterilizing factor, and a water supplier (20) arranged above the sterilized water producing section (10).

˜Sterilized Water Producing Section˜The sterilized water producing section (10) includes, as shown in FIG. 3, a water treatment vessel (13) made of an electrically insulating material, a first electrode (16) and a second electrode (17) which are fixed in the casing (1), and a divider (11) which divides a space in the water treatment vessel (13) into a portion in which the first electrode (16) is immersed in water, and a portion in which the second electrode (17) is immersed in water.

˜Water Treatment Vessel˜

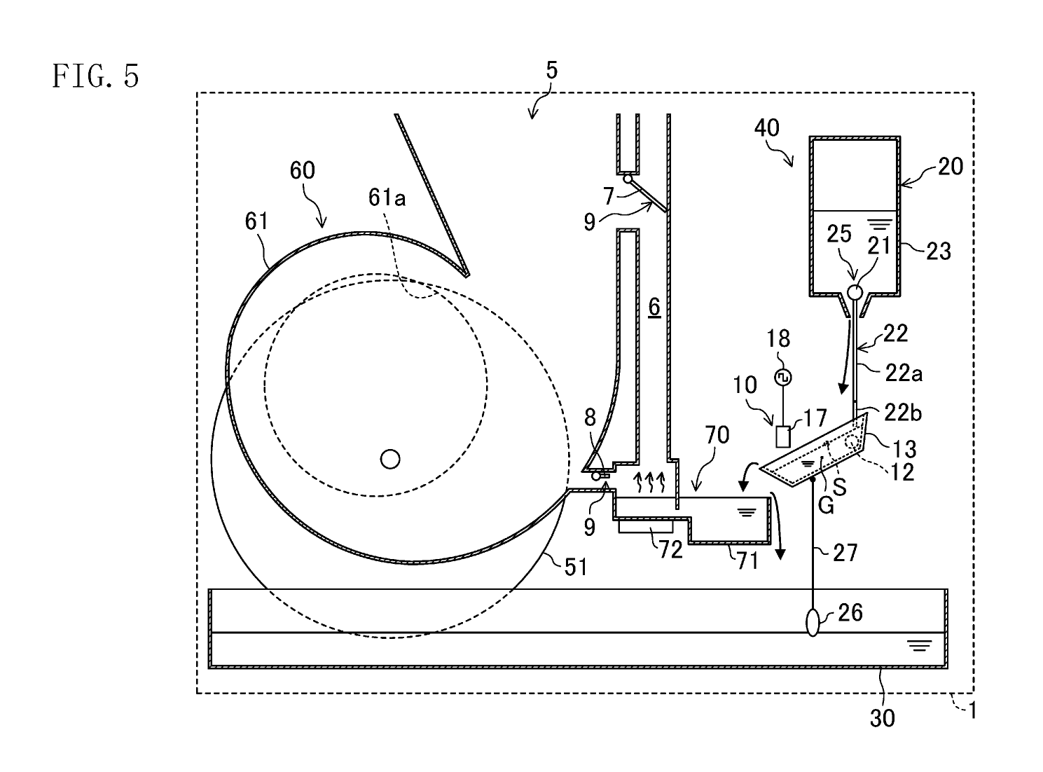

The water treatment vessel (13) is configured to cause electric discharge in water stored therein to produce a sterilizing factor. The water treatment vessel (13) is a box-shaped water tank having the shape of a boat when viewed from the side as shown in FIG. 2. The water treatment vessel (13) is supported by the casing (1) so that the water treatment vessel (13) is rotatable about a rotational shaft (S) parallel to the horizontal plane as shown in FIG. 2. Further, a floating arm (27) having a float (26) at the tip end thereof is rotatably attached to the bottom of the water treatment vessel (13) as shown in FIG. 2. The water treatment vessel (13) is configured to rotate in accordance with the level of water stored in a reservoir (30) to be described later such that the water treatment vessel (13) is switched between a discharging state shown in FIG. 2 and a draining state shown in FIG. 5, for example.

While the water treatment vessel (13) is in the discharging state as shown in FIG. 2, the water treatment vessel (13) and an auxiliary reservoir (71) to be described later have no communication, and the first and second electrodes (16) and (17) are immersed in water stored in the water treatment vessel (13) to cause electric discharge in the water. While the water treatment vessel (13) is in the draining state as shown in FIG. 5, the first and second electrodes (16) and (17) fixed in the casing (1) come out of water stored in the water treatment vessel (13) to stop the discharge in the water, and the water flows out of the water treatment vessel (13) into the auxiliary reservoir (71).

The water treatment vessel (13) is configured to be switched to the draining state if the level of water stored in the reservoir (30) is equal to or less than a predetermined value, and tilted in the draining state such that the water flows out of the water treatment vessel (13) over an edge thereof into the auxiliary reservoir (71) as shown in FIG. 5.

The rotational shaft (S) of the water treatment vessel (13) is misaligned from the center of gravity (G) of the water treatment vessel (13). Specifically, the rotational shaft (S) of the water treatment vessel (13) is located above the center of gravity (G) of the water treatment vessel (13). Further, the rotational shaft (S) of the water treatment vessel (13) is arranged opposite to the first and second electrodes (16) and (17) relative to the center of gravity (G) of the water treatment vessel (13).

˜Electrodes˜

The first and second electrodes (16) and (17) each have the shape of a flat plate, and are arranged to face each other. The first and second electrodes (16) and (17) are made of, for example, a metallic material which is highly resistant to corrosion. The first and second electrodes (16) and (17) are connected to a high voltage generator (18) as shown in FIG. 3. The high voltage generator (18) is comprised of a power source which applies a predetermined voltage to the first and second electrodes (16) and (17). Further, the high voltage generator (18) is configured to apply a voltage having a waveform alternating between positive and negative, for example, to the first and second electrodes (16) and (17).

˜Divider˜

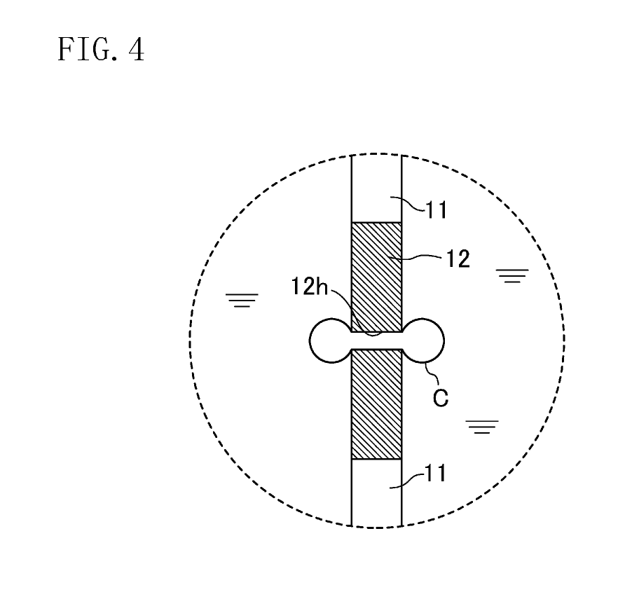

The divider (11) is made of an electrically insulating material. A disc-shaped discharging member (12) made of an electrically insulating material, such as ceramics or any other suitable material, is fitted in the divider (11) as shown in FIG. 4. Further, the discharging member (12) has a minute through hole (12h) having a diameter of about 50 μm, for example, which forms a current path between the first and second electrodes (16) and (17). The through hole (12h) is arranged to be always below the surface of water stored in the water treatment vessel (13) during the discharging state in which the first and second electrodes (16) and (17) are immersed in the water stored in the water treatment vessel (13). Further, the rotational shaft (S) penetrates the divider (11) as shown in FIG. 3. The divider (11) may be taller than the sidewalls of the water treatment vessel (13).

˜Water Supplier˜

The water supplier (20) is configured to supply water to the sterilized water producing section (10). As shown in FIG. 2, the water supplier (20) includes a water tank (23) which stores tap water, and a valve mechanism (25) provided at the bottom of the water tank (23). Further, the water supplier (20) is configured to supply water to the water treatment vessel (13) while the water treatment vessel (13) is in the draining state, and stop the supply of water while the water treatment vessel (13) is in the discharging state, depending on the state of the valve mechanism (25).

The water tank (23) is configured to be placed in, and removed from, the casing (1) from the side surface of the casing (1) (in the direction perpendicular to the paper of FIG. 1). Specifically, the water tank (23) is detachably housed in the casing (1). The bottom of the water tank (23) has a conical portion having a diameter gradually decreasing toward an outlet as shown in FIGS. 2 and 3.

The valve mechanism (25) is configured to be closed while the water treatment vessel (13) is in the discharging state, and opened while the water treatment vessel (13) is in the draining state. The valve mechanism (25) includes a spherical plug member (21) housed in the bottom of the water tank (23), and a water supply control rod (22) which displaces the plug member (21) at the bottom of the water tank (23) as shown in FIGS. 2 and 3. The water supply control rod (22) includes, as shown in FIG. 3, a push-up member (22a) which moves vertically to come into contact with the plug member (21), and a coupling member (22b) which is rotatably coupled with the push-up member (22a) and the divider (11).

The valve mechanism (25) is configured to move the push-up member (22a) downward to block the outlet of the water tank (23) with the plug member (21) as shown in FIG. 2 while the water treatment vessel (13) is in the discharging state, and move the push-up member (22a) upward to open the outlet of the water tank (23) as shown in FIG. 5 while the water treatment vessel (13) is in the draining state. When the water tank (23) is removed, the valve mechanism (25) allows the plug member (21) to remain in the water tank (23), and allows the water supply control rod (22) comprised of the push-up member (22a) and the coupling member (22b) to be coupled with the water treatment vessel (13) rotatably supported in the casing (1).

<Humidification Section>

The humidification section (50) includes, as shown in FIG. 1, a reservoir (30) placed on the bottom of the casing (1), and a humidification rotor (51) arranged such that a lower portion thereof is immersed in water stored in the reservoir (30).

The reservoir (30) is configured to store water supplied from the auxiliary reservoir (71). The reservoir (30) is configured to be placed in, and removed from, the casing (1) from the side surface of the casing (1) (in the direction perpendicular to the paper of FIG. 1).

The humidification rotor (51) is comprised of a disc-shaped member made of water-absorptive nonwoven fabric, for example. Further, the humidification rotor (51) is configured to be driven to rotate by a driving motor (not shown).

<Ultrasonic Irradiation Unit>

As shown in FIG. 2, the ultrasonic irradiation unit (70) includes an auxiliary reservoir (71) which stores sterilized water flowing from the water treatment vessel (13) of the water treatment unit (40), and a ultrasonic irradiator (72) which atomizes the sterilized water stored in the auxiliary reservoir (71) through ultrasonic irradiation of the sterilized water to produce sterilizing mist.

The bottom of the auxiliary reservoir (71) is stepped to have an upper bottom portion and a lower bottom portion. The ultrasonic irradiator (72) is attached to the upper bottom portion of the auxiliary reservoir (71). The lower bottom portion of the auxiliary reservoir (71) is configured to receive the sterilized water flowing from the water treatment vessel (13), and allow the sterilized water to flow over an edge of the auxiliary reservoir (71) into the reservoir (30), so that the level of water in the upper bottom portion of the auxiliary reservoir (71) is equal to or less than a predetermined level. Further, the upper bottom portion of the auxiliary reservoir (71) communicates with the auxiliary passage (6) through which the sterilizing mist produced by the ultrasonic irradiator (72) is delivered to the outside.

The ultrasonic irradiator (72) includes, for example, a pair of metallic plates, and a piezoelectric ceramic member sandwiched between the pair of metallic plates, and is configured to produce ultrasonic waves having a frequency of about 1 MHz or more, for example, if an alternating voltage is applied between the pair of metallic plates.

<Fan>

The fan (60) is configured to suck the air in the room through the inlet (2), and produce in the air passage (5) an air flow delivering the humidified air to the outside to blow the humidified air to a relatively wide region of the room through the primary outlet (3), and simultaneously, produce in the auxiliary passage (6) an air flow delivering the sterilizing mist to the outside to blow the sterilizing mist to a relatively narrow region of the room through the secondary outlet (4).

The fan (60) includes, as shown in FIG. 2, a fan casing (61), and an impeller (not shown) rotatably provided in the fan casing (61). The fan casing (61) has an inlet (61a) through which the humidified air produced in the humidification section (50) is sucked, and an outlet which communicates with the primary outlet (3) of the casing. The fan (60) may be, for example, a sirocco fan.

˜Operation Mechanism˜An operation mechanism of the air purifier (80) of the present embodiment will now be described below. In this air purifier (80), basically, the water treatment unit (40) performs a water purification operation, the ultrasonic irradiation unit (70) performs an atomization operation with water purified by the water treatment unit (40), and the humidification rotor (51) performs a humidification operation with water overflowed from the ultrasonic irradiation unit (70) while the air purification unit (45) purifies the air in the room.

<Water Purification Operation>

For the water purification operation, the reservoir (30) and the water tank (23) filled with tap water in advance are placed in the casing (1). Further, the high voltage generator (18) applies a predetermined voltage to the first and second electrodes (16) and (17).

When the reservoir (30) and the water tank (23) are placed, the water treatment vessel (13) in the reservoir (30) is tilted by its weight as shown in FIG. 5, and simultaneously, the water supply control rod (22) moves upward. As a result, the valve mechanism (25) is opened, and the water supplier (20) supplies water to the water treatment vessel (13). In this state, the water treatment vessel (13) is tilted. Thus, most of water supplied to the water treatment vessel (13) flows out of the water treatment vessel (13), and is stored in the auxiliary reservoir (71).

Water continuously flows into the auxiliary reservoir (71), and then overflows from the auxiliary reservoir (71) into the reservoir (30). Then, the level of water in the reservoir (30) rises, the float (26) moves upward, and the water treatment vessel (13) rotates clockwise about the rotational shaft (S). As a result, the amount of tilt of the water treatment vessel (13) gradually decreases, and water is stored in the water treatment vessel (13).

As the amount of water stored in the reservoir (30) increases, the water treatment vessel (13) further rotates clockwise until the water treatment vessel (13) is maintained in a horizontal position as shown in FIG. 2. Thus, the water treatment vessel (13) and the auxiliary reservoir (71) no longer communicate with each other, and the first and second electrodes (16) and (17) are immersed in water stored in the water treatment vessel (13), thereby causing electric discharge in the water. In this state, the water treatment vessel (13) is switched from the draining state to the discharging state. When the water supply control rod (22) moves downward along with the rotation of the water treatment vessel (13), the valve mechanism (25) is closed, and the water supplier (20) stops the supply of water to the water treatment vessel (13).

When the first and second electrodes (16) and (17) are immersed in the water stored in the water treatment vessel (13), a current density of the current path formed by the through hole (12h) in the discharging member (12) of the divider (11) increases.

The increase in current density of the current path in the through hole (12h) increases Joule heat generated in the through hole (12h). As a result, water is vaporized inside, and around the openings, of the through hole (12h) of the discharging member (12), thereby forming a bubble (C) in the through hole (12h). This bubble (C) covers the entire region of the through hole (12h) as shown in FIG. 4. In this state, the bubble (C) functions as a resistance that inhibits electrical conduction between the first and second electrodes (16) and (17) via water. Thus, the potential difference between the first and second electrodes (16, 17) and water becomes almost zero, and an interface between the bubble (C) and water on one side of the divider (11) and an interface between the bubble (C) and water on the other side of the divider (11) serve as electrodes. Then, dielectric breakdown occurs in the bubble (C), thereby causing electric discharge.

If the electric discharge occurs in the bubble (C) in this manner, a sterilizing factor, such as hydroxyl radicals, is produced in the water stored in the water treatment vessel (13), thereby producing sterilized water containing the sterilizing factor.

Further, if a humidification operation to be described later is performed, the amount of water in the reservoir (30) decreases to lower the level of water in the reservoir (30). Then, the water treatment vessel (13) is switched from the discharging state (see FIG. 2) to the draining state (see FIG. 5). Moreover, if the sterilized water is supplied from the water treatment vessel (13) to the reservoir (30) to raise the level of water in the reservoir (30), the water treatment vessel (13) is switched from the draining state (see FIG. 5) to the discharging state (see FIG. 2). In this way, the water treatment vessel (13) rotates if the level of water in the reservoir (30) changes, and is automatically switched between the draining state (see FIG. 5) and the discharging state (see FIG. 2).

In this manner, the water purification operation is performed. When the water treatment vessel (13) is switched from the discharging state to the draining state, the sterilized water stored in the water treatment vessel (13) and containing the sterilizing factor is supplied to the auxiliary reservoir (71), and the atomization operation is performed with the sterilized water.

<Atomization Operation>

During the atomization operation, the sterilized water from the water treatment vessel (13) is stored in the auxiliary reservoir (71), and an alternating voltage is applied between the pair of metallic plates of the ultrasonic irradiator (72). When the alternating voltage is applied between the pair of metallic plates of the ultrasonic irradiator (72), the sterilized water in the auxiliary reservoir (71) is irradiated with ultrasonic waves. Through the ultrasonic irradiation of the sterilized water in the auxiliary reservoir (71), hydrogen peroxide in the sterilized water turns to hydroxyl radicals, and the sterilized water is atomized to produce sterilizing mist.

In this manner, the atomization operation of the sterilized water is performed. The sterilized water overflowed from the auxiliary reservoir (71) is supplied to the reservoir (30) and used for humidification of the air.

<Humidification Operation>

During the humidification operation, the fan (60) is operated, and the humidification rotor (51) is driven to rotate by a driving motor. Further, a voltage is applied between the electrodes of the ionizer (42).

When the fan (60) is operated, the air in the room is introduced into the air passage (5) in the casing (1) through the inlet (2). The air introduced into the air passage (5) passes through the prefilter (41), during which some of dust particles are removed from the air, and further passes through the ionizer (42). The ionizer (42) causes corona discharge between the electrodes, thereby charging the dust particles in the air. The air that has passed through the ionizer (42) passes through the electrostatic filter (43). The electrostatic filter (43) electrically attracts and traps the dust particles charged by the ionizer (42). The air that has passed through the electrostatic filter (43) flows through the deodorizing filter (44). The deodorizing filter (44) removes target components (odor and toxic substances) from the air. The air that has passed through the deodorizing filter (44) passes through the humidification rotor (51).

Water stored in the reservoir (30) is absorbed on a rotor body of the humidification rotor (51). When the air passes through the humidification rotor (51), moisture adsorbed on the humidification rotor (51) is released in the air. In this manner, the humidification operation is performed.

<Operation Switching Mechanism>

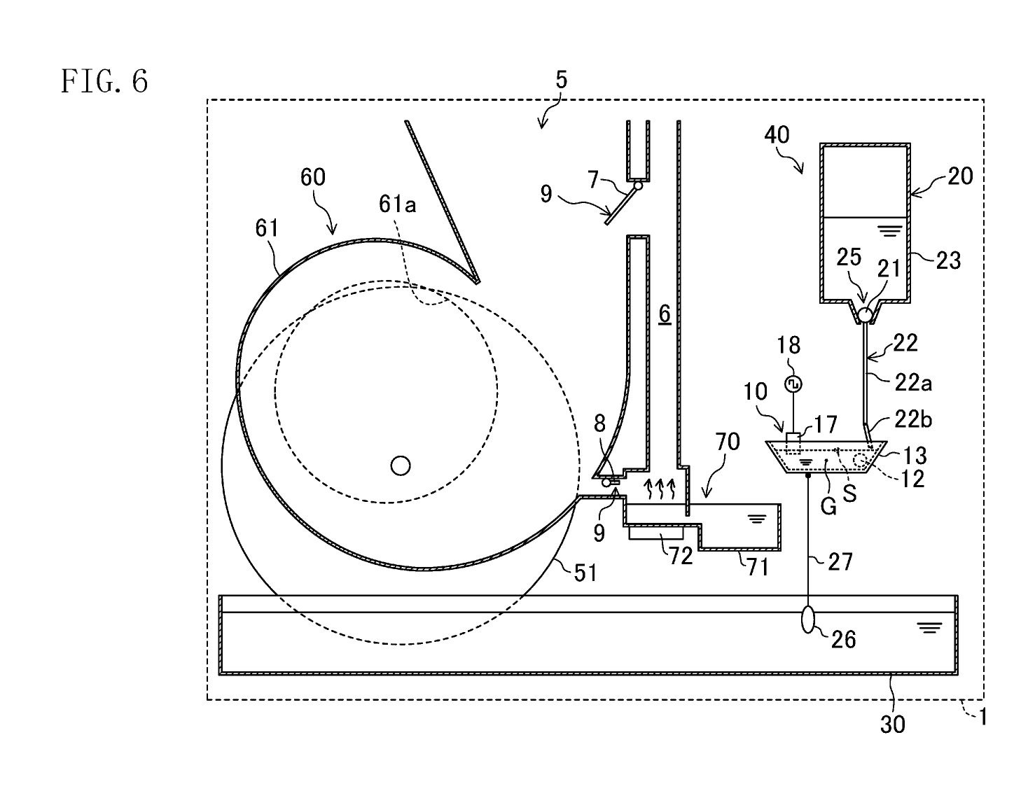

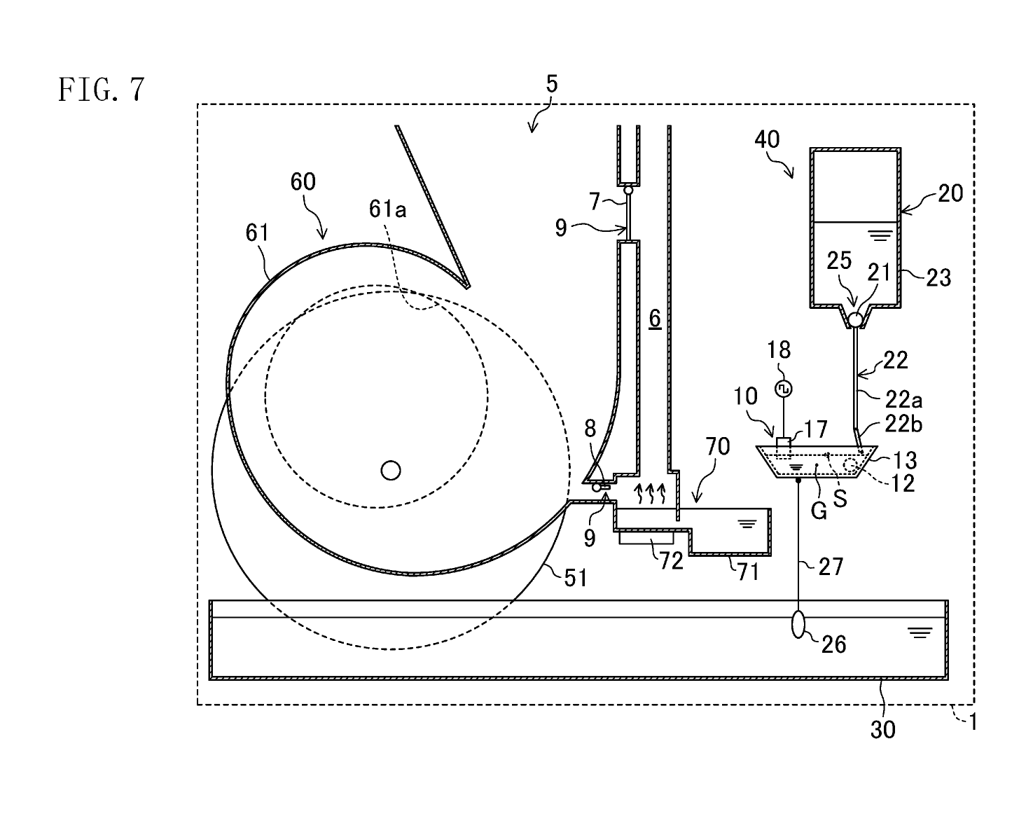

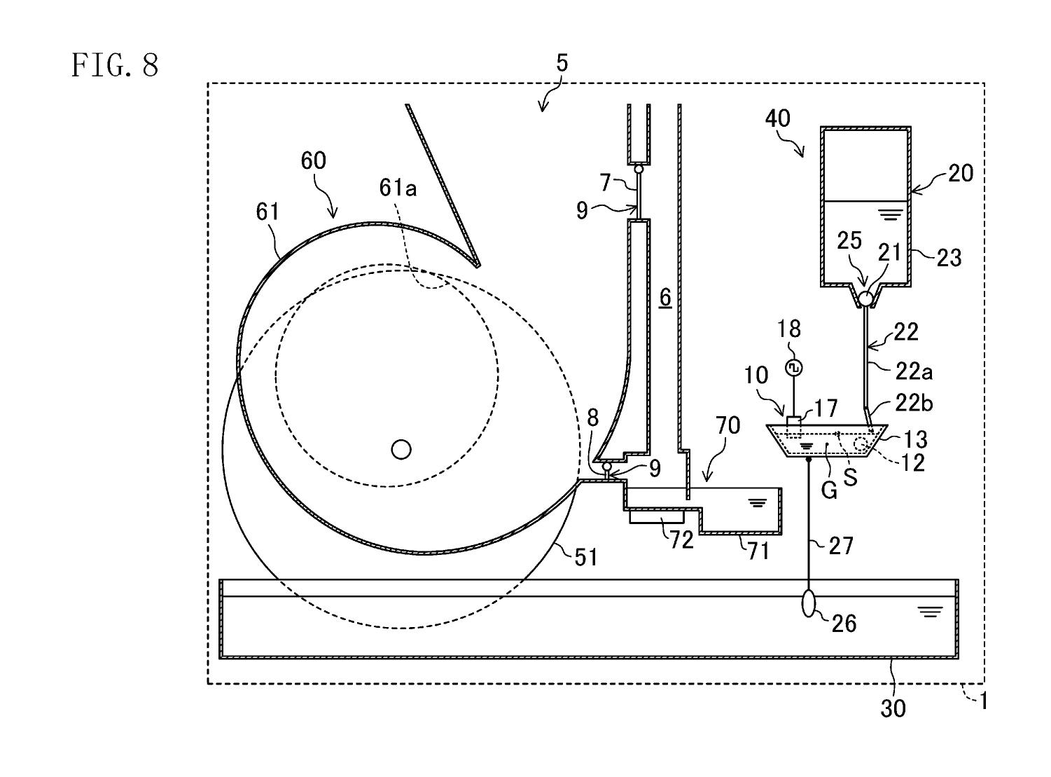

The operation switching mechanism (9) of the air purifier (80) is switched among a first state of a first operation mode (see FIG. 2), a second state of the first operation mode (see FIG. 6), a second operation mode (see FIG. 7), and a third operation mode (see FIG. 8).

The operation switching mechanism (9) in the first state of the first operation mode allows the inlet valve (8) to open, and the outlet valve (7) to open toward the auxiliary passage (6) as shown in FIG. 2, so that the sterilizing mist in the auxiliary passage (6) is introduced into the air passage (5). In this state, the humidification operation and the atomization operation are performed, and the flow of the humidified air introduced via the inlet valve (8) produces an air flow including the sterilizing mist in the auxiliary passage (6). The air flow including the sterilizing mist is introduced into the air passage (5) via the outlet valve (7). Thus, the humidified air blown from the air passage (5) into the room increases in absolute humidity.

The operation switching mechanism (9) in the second state of the first operation mode allows the inlet valve (8) to open, and the outlet valve (7) to open toward the air passage (5) as shown in FIG. 6, so that the humidified air in the air passage (5) is introduced into the auxiliary passage (6). In this state, the humidification operation and the atomization operation are performed, and the flow of the humidified air introduced via the inlet valve (8) produces an air flow including the sterilizing mist in the auxiliary passage (6). The air flow including the sterilizing mist in the auxiliary passage (6) is accelerated by the flow of the humidified air introduced via the outlet valve (7). Thus, the sterilizing mist is vigorously blown from the auxiliary passage (6) into the room, thereby bringing the sterilizing mist to a position away from the air purifier (80).

The operation switching mechanism (9) in the second operation mode allows the inlet valve (8) to open, and the outlet valve (7) to close as shown in FIG. 7. In this state, the humidification operation and the atomization operation are performed, and the flow of the humidified air introduced via the inlet valve (8) produces an air flow including the sterilizing mist in the auxiliary passage (6). Then, the sterilizing mist is released from the auxiliary passage (6) to a relatively narrow region of the room together with the humidified air, and the humidified air is released from the air passage (5) to a relatively wide region of the room. Thus, if hands or articles, for example, are placed in the flow of the sterilizing mist blown from the auxiliary passage (6), the hands or articles may be sterilized.

The operation switching mechanism (9) in the third operation mode allows the inlet valve (8) and the outlet valve (7) to close as shown in FIG. 8. In this state, the atomization operation is not performed, and only the humidified air is released from the air passage (5) to the relatively wide region of the room.

In this manner, the operation modes of the air purifier (80) may be switched.

˜Advantages of Embodiments˜

As can be seen from the foregoing, according to the air purifier (80) of the present embodiment, the fan (60) produces in the air passage (5) the air flow delivering the humidified air produced in the humidification section (50) to the outside. Further, the ultrasonic irradiator (72) atomizes the sterilized water produced by the sterilized water producing section (10) through ultrasonic irradiation of the sterilized water to produce the sterilizing mist.

In the first operation mode of the operation switching mechanism (9), the air passage (5) communicates with the auxiliary passage (6) through which the sterilized water is delivered to the outside so as to introduce the sterilizing mist into the air passage (5). Thus, the sterilizing mist in the auxiliary passage (6) is released from the air passage (5) to the outside together with the humidified air in the air passage (5). Further, in the second operation mode of the operation switching mechanism (9), the communication between the air passage (5) and the auxiliary passage (6) through which the sterilized water is delivered to the outside is blocked. Thus, the sterilizing mist in the auxiliary passage (6) is released from the auxiliary passage (6) to the outside.

Thus, mixing the sterilizing mist produced by atomization of the sterilized water with the humidified air improves the humidification capacity. In addition, the sterilizing mist may be supplied from the auxiliary passage (6) to a relatively narrow region of the room so that the humidifier may be used for various other purposes (e.g., sterilization of hands or articles). Therefore, according to the present embodiment, the air purifier (80) having the humidification function may be provided with improved convenience.

Moreover, according to the air purifier (80) of the present embodiment, the operation switching mechanism (9) includes the outlet valve (7) and the inlet valve (8) which allow the air passage (5) and the auxiliary passage (6) to communicate with each other as needed. Thus, opening/closing the outlet and inlet valves (7) and (8) of the operation switching mechanism (9) enables the switching between the first and second operation modes. Furthermore, the fan (60), which produces in the air passage (5) the air flow delivering the humidified air to the outside, produces in the auxiliary passage (6) the air flow delivering the sterilizing mist to the outside. Thus, the air flow delivering the humidified air to the outside and the air flow delivering the sterilizing mist to the outside are produced by the single fan (60).

In addition, according to the air purifier (80) of the present embodiment, the operation switching mechanism (9) in the first operation mode is configured to allow the inlet valve (8) to open, and the outlet valve (7) to open toward the auxiliary passage (6), so that the sterilizing mist in the auxiliary passage (6) is introduced into the air passage (5). Thus, the air flow including the sterilizing mist is produced in the auxiliary passage (6) by the flow of the humidified air introduced through the inlet valve (8), and is introduced into the air passage (5) through the outlet valve (7). Consequently, the sterilizing mist is mixed with the humidified air to increase the absolute humidity of the humidified air, and the room is reliably humidified by the humidified air blown into the room.

Further, according to the air purifier (80) of the present embodiment, the operation switching mechanism (9) in the first operation mode is configured to allow the inlet valve (8) to open, and the outlet valve (7) to open toward the air passage (5), so that the humidified air in the air passage (5) is introduced into the auxiliary passage (6). The flow of the humidified air introduced through the inlet valve (8) produces the air flow including the sterilizing mist in the auxiliary passage (6). Furthermore, the air flow including the sterilizing mist in the auxiliary passage (6) is accelerated by the flow of the humidified air introduced via the outlet valve (7). Thus, the sterilizing mist is vigorously blown from the auxiliary passage (6) into the room, thereby bringing the sterilizing mist to a position away from the air purifier (80).

According to the present embodiment, the operation switching mechanism (9) in the second operation mode allows the inlet valve (8) to open, and the outlet valve (7) to close. Thus, the flow of the humidified air introduced via the inlet valve (8) produces the air flow including the sterilizing mist in the auxiliary passage (6). As a result, the sterilizing mist may be released from the auxiliary passage (6) to a relatively narrow region of the room together with the humidified air.

On top of that, according to the air purifier (80) of the present embodiment, the sterilized water producing section (10) includes a water treatment vessel (13) which stores water, a first electrode (16) and a second electrode (17) which are provided to be immersed in water in the water treatment vessel (13), and a divider (11) which divides a space in the water treatment vessel (13) into a portion in which the first electrode (16) is immersed in water, and a portion in which the second electrode (17) is immersed in water. The discharging member (12) of the divider (11) has a through hole (12h) which forms a current path between the first and second electrodes (16) and (17). Thus, when a voltage is applied between the first and second electrodes (16) and (17), a bubble is formed in water stored in the water treatment vessel (13), more specifically, in the through hole (12h) in the discharging member (12) to serve as the current path between the first and second electrodes (16) and (17), and electric discharge occurs in the bubble. As a result, a sterilizing factor, such as hydroxyl radicals, may be produced in the water stored in the water treatment vessel (13). The hydroxyl radicals produced in the water in the water treatment vessel (13) shortly turn to be hydrogen peroxide. However, the hydrogen peroxide is decomposed and hydroxyl radicals are produced again through ultrasonic irradiation by the ultrasonic irradiator (72). Thus, the sterilizing mist containing hydroxyl radicals may be released into the room.

<<Other Embodiments>>

In the first embodiment described above, the air purifier including the water treatment vessel (13) which causes electric discharge in water stored therein has been described. However, the present invention is also applicable to an air purifier including a water treatment vessel which causes electrolysis in water stored therein.

Note that the above-described embodiments are merely exemplary ones in nature, and do not intend to limit the scope, application or uses of the present invention.

### INDUSTRIAL APPLICABILITY

As can be seen from the foregoing, the present invention is useful for a humidifier which produces humidified air.

### DESCRIPTION OF REFERENCE CHARACTERS

5 Air Passage
6 Auxiliary Passage
7 Outlet Valve
8 Inlet Valve
9 Operation Switching Mechanism
10 Sterilized Water Producing Section
11 Divider
12h Through Hole
13 Water Treatment Vessel
16 First Electrode (One of a Pair of Electrodes)
17 Second Electrode (The Other of the Pair of Electrodes)
50 Humidification Section
60 Fan
72 Ultrasonic Irradiator
80 Air Purifier (Humidifier)

1. A humidifier comprising:
a humidification section which vaporizes water stored therein to produce humidified air;
an air passage through which the humidified air is delivered to the outside; and
a fan which produces in the air passage an air flow delivering the humidified air to the outside, wherein
the humidifier further comprises: a sterilized water producing section which produces a sterilizing factor in water stored therein to produce sterilized water containing the sterilized factor,
an ultrasonic irradiator which atomizes the sterilized water through ultrasonic irradiation of the sterilized water to produce sterilizing mist,
an auxiliary passage through which the sterilizing mist is delivered to the outside; and
an operation switching mechanism which is switched between a first operation mode in which the air passage and the auxiliary passage communicate with each other to introduce the sterilizing mist into the air passage, and a second operation mode in which communication between the air passage and the auxiliary passage is blocked.

2. The humidifier of claim 1, wherein
the operation switching mechanism includes an outlet valve which allows or blocks communication between an end of the air passage adjacent to an outlet and an end of the auxiliary passage adjacent to an outlet, and an inlet valve which allows or blocks communication between an end of the air passage upstream of the outlet valve and an end of the auxiliary passage adjacent to the ultrasonic irradiator, and
the fan is configured to produce in the auxiliary passage an air flow delivering the sterilizing mist to the outside.

3. The humidifier of claim 2, wherein
the operation switching mechanism in the first operation mode allows the inlet valve to open, and the outlet valve to open toward the auxiliary passage, so that the sterilizing mist in the auxiliary passage is introduced into the air passage.

4. The humidifier of claim 2, wherein
the operation switching mechanism in the first operation mode allows the inlet valve to open, and the outlet valve to open toward the air passage, so that the humidified air in the air passage is introduced into the auxiliary passage.

5. The humidifier of claim 2, wherein
the operation switching mechanism in the second operation mode allows the inlet valve to open, and the outlet valve to close.

6. The humidifier of claim 1, wherein
the sterilized water producing section includes: a water treatment vessel which stores water; a pair of electrodes which are provided to be immersed in water in the water treatment vessel; and a divider which is made of an electrically insulating material, and divides a space in the water treatment vessel into a portion in which one of the pair of electrodes is immersed in water, and a portion in which the other of the pair of electrodes is immersed in water, a through hole is formed through the divider to form a current path between the pair of electrodes, and electric discharge is caused in the through hole in water to produce the sterilizing factor.

7. The humidifier of claim 2, wherein
the sterilized water producing section includes: a water treatment vessel which stores water; a pair of electrodes which are provided to be immersed in water in the water treatment vessel; and a divider which is made of an electrically insulating material, and divides a space in the water treatment vessel into a portion in which one of the pair of electrodes is immersed in water, and a portion in which the other of the pair of electrodes is immersed in water, a through hole is formed through the divider to form a current path between the pair of electrodes, and electric discharge is caused in the through hole in water to produce the sterilizing factor.

8. The humidifier of claim 3, wherein
the sterilized water producing section includes: a water treatment vessel which stores water; a pair of electrodes which are provided to be immersed in water in the water treatment vessel; and a divider which is made of an electrically insulating material, and divides a space in the water treatment vessel into a portion in which one of the pair of electrodes is immersed in water, and a portion in which the other of the pair of electrodes is immersed in water, a through hole is formed through the divider to form a current path between the pair of electrodes, and electric discharge is caused in the through hole in water to produce the sterilizing factor.

9. The humidifier of claim 4, wherein
the sterilized water producing section includes: a water treatment vessel which stores water; a pair of electrodes which are provided to be immersed in water in the water treatment vessel; and a divider which is made of an electrically insulating material, and divides a space in the water treatment vessel into a portion in which one of the pair of electrodes is immersed in water, and a portion in which the other of the pair of electrodes is immersed in water, a through hole is formed through the divider to form a current path between the pair of electrodes, and electric discharge is caused in the through hole in water to produce the sterilizing factor.

10. The humidifier of claim 5, wherein
the sterilized water producing section includes: a water treatment vessel which stores water; a pair of electrodes which are provided to be immersed in water in the water treatment vessel; and a divider which is made of an electrically insulating material, and divides a space in the water treatment vessel into a portion in which one of the pair of electrodes is immersed in water, and a portion in which the other of the pair of electrodes is immersed in water, a through hole is formed through the divider to form a current path between the pair of electrodes, and electric discharge is caused in the through hole in water to produce the sterilizing factor.
